(19) United States
(12) Patent Application Publication
(10) Pub. No.: US 2026/0321427 A1
(43) Pub. Date: Oct. 15, 2026

(54) COMPUTER-IMPLEMENTED METHOD AND SYSTEM FOR DETERMINING AN ACUTE MYOCARDIAL INFARCTION TYPE INDICATOR

(71) Applicant: BECKMAN COULTER, INC., Brea, CA (US)

(72) Inventors: Amy Karen KRAJECK, Prior Lake, MN (US); Max SHATSKY, Castro Valley, CA (US)

(21) Appl. No.: 19/164,563

(22) PCT Filed: Apr. 2, 2024
(86) PCT No.: PCT/US2024/022649

§ 371 Date: Sep. 11, 2025

Related U.S. Application Data
(60) Provisional application No. 63/457,502, filed on Apr. 6, 2023.

(51) Int. Cl. G16H 50/30; A61B 5/00

(52) U.S. Cl. CPC G16H 50/30; A61B 5/7275

(57) ABSTRACT

A computer-implemented method of determining an acute myocardial infarction, AMI, type indicator, is described. The method comprises obtaining, at a computing device, subject data associated with a subject suspected of having AMI, the subject data comprising a) at least one Troponin value indicative of a Troponin concentration determined for the subject, b) at least one Albumin value indicative of an Albumin concentration determined for the subject, and c) at least one Eosinophil value indicative of an Eosinophil concentration determined for the subject. The method further comprises computing, based on the obtained subject data with the computing device, at least one AMI type indicator indicative of one or both a likelihood for the subject to have a type 1 AMI and a likelihood for the subject to have a type 2 AMI.

### CROSS-REFERENCE TO RELATED APPLICATIONS

This application claims the benefit of priority of U.S. Provisional Application Ser. No. 63/457,502, filed Apr. 6, 2023, which is hereby incorporated by reference in its entirety.

### TECHNICAL FIELD

The present disclosure generally relates to the field of clinical decision support. In particular, the present disclosure relates to a computer-implemented method of determining an Acute Myocardial Infarction, AMI, type indicator indicative of a type of AMI based on data associated with a subject, for example a subject, patient or individual suspected of having AMI. The present disclosure also relates to a computing device or system configured to carry out steps of said method, to a corresponding computer program instructing the computing device or system to perform steps of said method, and to a computer-readable medium storing such computer program.

### BACKGROUND

One of the most frequent causes of adult emergency department (ED) visits is chest pain, which is also a common symptom associated with acute myocardial infarction (AMI, also referred to as heart attack). About 10% of these patients will be diagnosed later with life-relevant conditions, such as acute coronary syndrome, a disease state that includes acute myocardial infarction.

AMI is a myocardial cell death caused by acute obstruction of a coronary artery. Earlier detection and treatment can restore blood oxygen supply and stop cell necrosis. As such, early diagnosis or rule-out of AMI at Emergency Departments and hospitals would be favorable, which can lead to rapid treatment as well as reduced cost and resource burden of both hospital and patient, for example by reducing the number of tests and leading to earlier discharges and improved risk stratification at hospitals.

The health state of a subject suspected of having AMI as well as the course of the disease over time can largely differ among different subjects or patients. The “Fourth Universal Definition of Myocardial Infarction (2018)” defines five types of acute myocardial infarction, named type 1 AMI through type 5 AMI. In particular, different criteria related to different symptoms and different treatments of patients having or suspected of having AMI are defined. According to this definition of classification of AMI, for example, AMI type 1, can be caused due to partial or complete blockage of the coronary arteries by a blood clot or the combination of a blood clot and arterial plaques. AMI type 2, on the other hand, is defined as being specifically not related to coronary atherothrombosis (blood clot), but is characterized by an imbalance in oxygen supply and demand. Similar criteria have been defined by the “Fourth Universal Definition of Myocardial Infarction (2018)” for AMI types 3 to 5, which is incorporated herein by reference, in particular regarding the definition and classification of AMI types 1 through 5.

As the different types of AMI may have different causes accompanied by different symptoms, different treatments may be required for patients depending on the AMI type. This particularly applies to type 1 and type 2 AMI patients, the latter being, for example, more likely to receive therapies for concomitant disease, such as receiving anti-coagulants, rather than being prepared for stent placement or by-pass surgery, as is typically the case for AMI type 1 patients. Hence, early differentiation between different types of AMI patients, in particular type 1 and type 2 AMI patients, at Emergency Departments and Hospitals would be desirable to benefit from earlier and more directed therapies. Among the patients diagnosed with AMI at EDs, approximately 75% have AMI Type 1 which typically requires revascularization treatment. The remaining 25% have AMI Type 2 which does not usually require surgical intervention, but may generally be associated with chronic disease and can be treated medically.

While the modern standard of care can diagnose myocardial infarction with high accuracy, e.g. using high sensitivity troponin tests, differentiating between different types of AMI is still challenging as typically would require invasive procedures like coronary angiography.

### SUMMARY

Therefore, it would be desirable to provide for a computational method and tool that allows for a determination of different types of AMI based on data or laboratory data associated with and available for a patient, subject or individual, for example a patient suspected of having AMI. In particular, it would be desirable to provide for an improved method and device for risk stratification and segregation of type 1 and type 2 acute myocardial infarction.

This is achieved by the subject matter of the independent claims. Exemplary embodiments are incorporated in the dependent claims and the following description.

An aspect of the present disclosure refers to a computer-implemented method of determining, for a patient subject or individual, an acute myocardial infarction, AMI, type indicator, indicative of a type of AMI. A further aspect of the present disclosure refers to a computing device or system including one or more processors for data processing, wherein the computing device or system is configured to carry out steps of the method described herein. A further aspect of the present disclosure refers to a computer program, which, when executed by one or more processors of a computing device or system, instructs the computing device or system to perform steps of the method described herein. Yet a further aspect of the present disclosure refers to a computer-readable medium, for example a non-transitory computer-readable medium, storing such computer program. Any disclosure presented herein with reference to one aspect of the present disclosure equally applies to any other aspect of the present disclosure, unless explicitly stated otherwise.

According to a first aspect of the present disclosure, there is provided a computer-implemented method of determining an acute myocardial infarction, AMI, type indicator and/or an indicator for a type of AMI. Alternatively or additionally, the method may relate to a computer-implemented method of determining a type of AMI for a subject, patient and/or individual, for example suspected of having AMI. Alternatively or additionally, the method may refer to a computer-implemented method of AMI-typing a subject, e.g. based on determining the AMI type indicator. Alternatively or additionally, the method may refer to a computer-implemented method of assessing and/or determining a risk, probability or likelihood of having a particular type of AMI. It is noted that while aspects of the present disclosure particularly refer to type 1 AMI and type 2 AMI (also referred to as AMI type 1 and AMI type 2).

The computer-implemented method of determining an AMI type indicator and/or an indicator for a type of AMI described herein comprises obtaining, at a computing device, subject data associated with a subject, patient and/or individual suspected of having AMI, wherein the subject data comprises:
a) at least one Troponin value, Tn value, indicative of a Troponin concentration determined for the subject,
b) at least one Albumin value indicative of an Albumin concentration determined for the subject, and
c) at least one Eosinophil value indicative of an Eosinophil concentration determined for the subject.

The method comprises computing, based on the obtained subject data with the computing device, at least one AMI type indicator indicative of one or both a likelihood for the subject to have a type 1 AMI and a likelihood for the subject to have a type 2 AMI.

Determining the at least one AMI type indicator in accordance with the method described herein can refer to AMI-typing, which may be synonymously used with determining or detecting an AMI type for the subject. The method described herein can allow for an accurate detection, differentiation and/or segregation of subjects or patients having type 1 AMI or type 2 AMI. Hence, the method and device described herein can assist healthcare providers (HCPs) with the early identification of the type of AMI to facilitate early intervention and appropriate treatment according to the particular type of AMI.

The inventors surprisingly found that taking at least one Troponin value, at least one Albumin value and at least one Eosinophil value of the patient or subject into consideration, the type of AMI can be determined and/or assessed, in particular it can be discerned whether the subject is more likely to have type 1 AMI or type 2 AMI. Alternatively or additionally, the method and device can allow to detect an AMI type, exclude an AMI type, detect a particular type of AMI and/or exclude a particular type of AMI for a particular subject. Accordingly, by means of the computer-implemented method described herein, risk stratification, for example at hospitals or emergency departments, can be significantly improved.

Moreover, by determining the AMI type indicator based on at least one Troponin value, at least one Albumin value and at least one Eosinophil value, a health state of the patient can be comprehensively examined or evaluated. As a consequence, the determination of the particular type of AMI a patient or subject suspected of having AMI actually has, can be largely individualized thereby improving overall accuracy of diagnosis and treatment.

The present disclosure, therefore, can provide for an improved clinical decision support system, for example allowing to efficiently, reliably and accurately risk stratify a patient, individual or subject as, for example, having type 1 AMI or type 2 AMI. For instance, aspects of the present disclosure may facilitate earlier discharge of patients from hospitals for patients having a low risk or being not at risk for AMI, and earlier intervention with the appropriate treatment for those patients who are at risk for either type 1 AMI or type 2 AMI. Hence, unnecessary hospitalization and anxiety among patients may be efficiently avoided or reduced. Also, certain procedures and protocols can be avoided at hospitals, which can result in a better patient experience, improved utilization of healthcare resources and cost savings, all while maintaining a high safety profile.

For instance, the method and device described herein can allow to reduce or avoid overcrowding at emergency departments. Also, the use of scarce healthcare resources can be optimized, such as observation beds, and diagnostic modalities, such as cardiac stress tests, angiograms and imaging studies. Further, the patient experience can be improved, for example by reducing the length of an emergency department stay. Alternatively or additionally, the documentation of risk assessment can be automated and the ability for shared decision-making with patients about diagnostic and treatment choices can be significantly improved.

It is emphasized that the computer-implemented method and device described herein relates to a data processing means for clinical decision support, and does not involve in and for itself any diagnostic or therapeutic activity.

As used herein, any parameter value of the subject or associated with the subject, for example any parameter value contained in the subject data, such as the at least one Troponin value, the at least one Albumin value and the at least one Eosinophil value, can be obtained based on a measurement, for example based on or using one or more samples from the patient, such as blood samples. This applies to any clinical data, subject data or patient data, such as vital signs, hematology parameters and/or pertinent laboratory results of the subject described herein as being usable to compute the at least AMI type indicator.

In particular, the at least one Troponin value, the at least one Albumin value, and the at least one Eosinophil value of the subject can relate to or be based on a measurement of the Troponin concentration, the Albumin concentration and the Eosinophil concentration, respectively, performed on the subject or patient, for example based on laboratory testing of sample material of the patient using one or more samples, such as blood samples. The same applies to any other parameter value described herein as being potentially usable for the computation of the one or more AMI type indicators.

It is emphasized that none of the parameter values or parameters referred to herein is limited to a particular type of measurement to obtain or measure the respective parameter value. For instance, the at least one Troponin value or Tn value may be based on any type of one or more troponin tests performed on the subject. For example, at least one Troponin value can relate to or be based on any one or more of a troponin I test (TnI), a high sensitivity troponin I test (hs-TnI), a troponin T test (TnT), and a high sensitivity troponin T test (hs-TnT). The same applies any other parameter referred to herein, including the one or more Albumin and Eosinophil values.

Optionally, the subject data may include time information for at least some of the parameter values included or indicated by the subject data. In particular, any one or more of the at least one Troponin value, the at least one Albumin value and the at least one Eosinophil value, can be associated with or include a time information, such as for example a timestamp. The same applies to any other parameter value described herein as being potentially usable for the computation of the at least AMI type indicator. The timestamp or time information associated with the one or more parameter values may indicate one or more of a measurement time the respective parameter value has been measured at the subject, a time of recordation of the parameter value, and/or a time of receipt of the parameter value at the computing device.

As used herein, determining the at least AMI type indicator may include computing and/or assessing the likelihood or probability for one or both AMI type 1 and AMI type 2. Any reference herein to a single AMI type indicator includes a plurality, unless stated otherwise.

For instance, determining may generally relate to or include finding out or coming to a decision about by the likelihood for the subject to have type 1 AMI and/or type 2 AMI, which may optionally include a calculation, a computation and/or a reasoning for the assessment of the AMI type indicator. Alternatively or additionally, computing the AMI type indicator may include determining based on mathematical means; algorithms and/or calculation the likelihood for the subject to have type 1 AMI and/or type 2 AMI. In particular, the determination of the likelihood for the subject to have type 1 AMI and/or type 2 AMI may be computer-aided, computer-assisted and/or computer-implemented.

As used herein, obtaining the subject data may comprise accessing said data and/or retrieving the subject data e.g. from the at least one memory of the computing device that carries out the method of the present disclosure, from the memory of another computing device, or from another remote data storage (a database, a secondary memory, a cloud storage or the like). Accordingly, in some cases, retrieving the subject data may comprise downloading said data. Additionally or alternatively, obtaining the subject data may comprise receiving said data, e.g. from a user or a computing device different from the computing device accessing the data. The two options are not mutually exclusive. For instance, obtaining the set of patient data may comprise receiving said set data, storing said set in the memory of the computer device and retrieving the set of patient data by accessing said memory. For instance, obtaining the subject data may comprise receiving the subject data at the computing device, for example at a data storage thereof. Alternatively or additionally, obtaining the subject data may include downloading, retrieving, and/or accessing the subject data by the computing device, for example at a data source remote from the computing device.

According to the method described herein, one or more AMI type indicators indicative of a likelihood or probability for at least one of type 1 AMI and type 2 AMI may be computed by the computing device based on the subject data. For instance, a single AMI type indicator may be computed, which may be indicative of either the likelihood for type 1 AMI or type 2 AMI. Alternatively or additionally, a single AMI type indicator may be computed that may be indicative of a relative likelihood for type 1 AMI and type 2 AMI, for example indicating a particular type to be more likely than the other. Alternatively or additionally, a plurality of type indicators may be computed. For example, for each of type 1 AMI and type 2 AMI, an AMI type indicator may be computed that may be indicative of the likelihood of the respective type of AMI. Alternatively or additionally, a plurality of AMI type indicators may be computed for a single type of AMI, in particular for one or both AMI type 1 and AMI type 2.

As used herein, the AMI type indicator may relate to a quantitative and/or qualitative measure indicative, representative and/or descriptive of a likelihood or probability for the subject to have type 1 AMI and/or a likelihood for the subject to have type 2 AMI. For example, the AMI type indicator can include or refer to a numerical measure or score on arbitrary scale, for example between zero and 100 or any other scale, indicative of the respective likelihood or probability for type 1 AMI and/or type 2 AMI. Alternatively or additionally, the type indicator may represent a particular risk level or tier for one or both type 1 AMI and type 2 AMI, such as low risk, medium risk, high risk. Alternatively or additionally, the AMI type indicator may indicate which of the types of AMI is more likely than the other.

In an embodiment, computing the at least one AMI type indicator further comprises classifying, by using or based on the subject data, the subject into at least one AMI type class of a plurality of AMI type classes, the plurality of AMI type classes comprising a type 1 AMI class of subjects suspected of having type 1 AMI and a type 2 AMI class of subjects suspected of having type 2 AMI. For instance, each class of the plurality of AMI type classes may be associated with a respective type of AMI. Generally, a classification or categorization in classes according to the respective AMI type can further improve segregation and risk stratification of subjects according to the actual AMI type they may be suspected to have.

For example, the classification into a plurality of AMI type classes can be based on the likelihood or probability for a particular subject to have a particular type of AMI considered in the plurality of AMI type classes. At least one of the plurality classes can be associated with a likelihood for the subject to have the particular AMI type, which AMI type class may then be selected as classification result indicating that the subject is likely to have the particular type of AMI associated with the respective AMI type class. Alternatively, a plurality of likelihoods may be computed and associated with a plurality of AMI type classes. The AMI type class with the highest likelihood may be then selected as classification result indicating that the particular subject is most likely having the AMI type of the selected AMI type class.

For the actual classification, the computing device may comprise a classifier, for example based on a Machine Learning model, as described in more detail hereinbelow.

According to an embodiment, the at least one AMI type indicator is indicative of a classification result of classifying the subject into at least one of a type 1 AMI class of subjects having type 1 AMI and a type 2 AMI class of subjects having type 2 AMI. The classification can for example relate or include a class label of the respective AMI type class, such AMI type 1 class or AMI type 2 class. Accordingly, the at least one AMI type indicator may represent or indicate a particular AMI type class, which the subject has been associated with based on the subject data and/or for which the highest probability among the plurality of AMI type classes has been determined based on the subject data. By providing a classification result indicating the type of AMI that, based on the classification result, a subject may have, interpretation of the at least AMI type indicator may be simplified, and erroneous interpretation may be avoided, for example when compared to numerical results or scores.

According to an embodiment, computing the at least one AMI type indicator includes computing the likelihood or probability for the subject to have type 1 AMI and the likelihood or probability for the subject to have type 2 AMI. Optionally, the method may comprise comparing the likelihood for the subject to have type 1 AMI and the likelihood for the subject to have type 2 AMI. For example, based on the comparison, the higher likelihood may be chosen and provided as AMI type indicator. Alternatively, at least two AMI type indicators may be provided, each associated with one of the determined likelihoods for AMI type 1 and AMI type 2, respectively. Alternatively, a single AMI type indicator may be computed or provided, which may be indicative of a difference between the computed likelihood for type 1 AMI and the computed likelihood for type 2 AMI. In other words, the AMI type indicator may indicate the relative likelihood of type 1 AMI and type 2 AMI. Exemplarily, a single AMI type indicator may be computed or provided, which may be indicative of a ratio between the computed likelihood for type 1 AMI and the computed likelihood for type 2 AMI.

According to an embodiment, computing the at least one AMI type indicator is based on at least one machine learning model, ML model, configured to receive and process input data associated with at least a subset of the subject data. The ML model may optionally provide as output the one or more AMI type indicators. For instance, the one or more AMI type indicators may be provided as classification result of the ML model.

In particular, some aspects of the present disclosure can use a predictive machine learning (ML) model to analyze relevant clinical data or subject data, such as vital signs or values, hematology parameters and/or laboratory results of a subject, to compute the at least AMI type indicator. The at least one machine learning model can, for example, comprise one or more of a trained logistic regression, a trained random forest, a trained gradient boosting algorithm, and a trained artificial neural network.

According to an embodiment, the at least one AMI type indicator is computed based on at least one machine learning model of the computing device configured to compute the at least one AMI type indicator based on a single or a plurality of Troponin value as input data. In other words, the same ML model may be used to compute the at least one AMI type indicator based on one Tn value or based on a plurality of Tn values obtained as subject data and for example provided as input data to the ML model.

Alternatively, a first ML model may be trained and configured to process a single Tn value as input data and a second ML model may be trained and configured to process a plurality of Tn values as input data and compute the AMI type indicator based thereon.

In an embodiment, computing the AMI type indicator is based on an obtained single Troponin value, wherein a further or updated AMI type indicator may be determined upon obtaining at least one further Troponin value for the subject. Alternatively or additionally, the AMI type indicator may be re-computed or updated upon obtaining at least one further Troponin value for the subject.

According to an embodiment, the subject data is indicative of a plurality of Troponin values, and the at least one AMI type indicator is computed based on the plurality of Tn values. Generally, each of the Tn values may be acquired or determined at a particular time point or time, wherein times or time points of different Tn values may be the same or may differ from one another. For example, different Tn tests can be performed at substantially the same time, or the same or different Tn tests can be performed at different times. In both scenarios, overall accuracy and performance of the computation of the AMI type indicator may be increased by taking a plurality of values into account.

In yet another embodiment, the at least one AMI type indicator is computed based on a plurality of Tn values, and one or both a plurality of Albumin values and a plurality of Eosinophil values. Analogue to the plurality of Tn values, also the plurality of Albumin and/or the plurality of Eosinophil values may be acquired and/or measured at same or different times. Also, for any other parameter value described herein as being usable for the computation of the AMI type indicator, a plurality of values may be used or obtained by the computing device.

As mentioned above, the plurality of values of a subject parameter, such as the plurality of Tn values, the plurality of Albumin values and/or the plurality of Eosinophil values, may be obtained at the same or different time points. For instance, according to the present disclosure, a plurality of values of a subject parameter (such as Tn, Albumin, Eosinophil) are obtained at different time point if, in particular, each value of the plurality of values of the subject parameter is obtained at a respective time points, in such a way that, for each pair of values of said plurality of values, the time point at which the first value of that pair of values is obtained.

In particular, the values of a plurality of values of a subject parameter, such as a plurality of Tn values, a plurality of Albumin values, a plurality of Eosinophil values or the like can be obtained at different time points, thereby allowing to take evolution of the parameters over time and optionally their effect or impact on the computed or re-computed AMI type indicator into account.

For instance, upon obtaining any further parameter value at the computing device, for example as soon as a new, updated or additional parameter value, such as a new updated or additional Tn value, is available in the subject data, the computing device may compute an updated AMI type indicator. This may allow to iteratively improve accuracy of the AMI type indicator.

The corresponding time information for each subject parameter value may be included in the subject data, for example in the form of a timestamp associated with the respective subject parameter value. Further, the timestamps or time information of different parameter values of a particular parameter may be identical, for example when two measurements of a parameter have been performed at the same time, or may differ, for example when two consecutive measurements of a parameter have been made.

According to an embodiment, obtaining the subject data includes obtaining a first Troponin value indicative of a first Troponin concentration determined for the subject at a first time, and obtaining a second Troponin value for the subject indicative of a second Troponin concentration determined for the subject at a second time different than the first time, wherein the AMI type indicator is computed based on the first Troponin value and based on the second Troponin value. As mentioned above, using a plurality of Tn values measured at different times at the subject, can reflect the course of an AMI and/or allow for a better differentiation between AMI type 1 and type 2. For instance, in case of AMI type 2, the Tn concentration in subject's blood may increase over time at least over a certain period of time, which can in turn be determined with higher accuracy when considering a plurality of Tn values at different times.

It should be noted though, that determining or computing the AMI type indicator based on a plurality of Tn values or other parameter values does not necessarily mean that all Tn values of the plurality of values are used for the actual computation of the AMI type indicator. Rather, the Tn values may be processed, and for example intercompared or filtered according to time and/or value, as will be described in more detail hereinbelow. For example, the plurality of values of a subject parameter (e.g. Tn, Albumin, Eosinophil) may be used to compute the input data of the trained ML model.

According to an embodiment, the method further comprises determining a maximum Troponin value among the obtained first Troponin value and second Troponin value (or determining the higher Tn value of the first and second Tn values), wherein the AMI type indicator is computed based on the determined maximum Troponin value. Likewise, a maximum Tn value may be determined among a plurality of more than two Tn values and used for computing the AMI type indicator. For instance, the Tn values may inter-compared and/or filtered according to determine the maximum Tn value. For instance, the Tn values may be inter-compared to determine the maximum Tn value. Also, it is noted that computing the AMI type indicator based on the maximum Tn value does not exclude that one or more of the other obtained Tn values, i.e., the Tn value(s) that are/is smaller than the maximum value, is used for computing the AMI type indicator. In particular, the maximum Troponin value may be the maximum of a first subset of values of the plurality of Troponin values, wherein the first subset of values consists of values of the plurality of Troponin values that are obtained in a given time interval.

According to an embodiment, the method further comprises determining a minimum Troponin value among the first Troponin value and second Troponin value (or determining the lower Tn value of the first and second Tn values), wherein the AMI type indicator is computed based on the determined minimum Troponin value. Likewise, a minimum Tn value may be determined among a plurality of more than two Tn values and used for computing the AMI type indicator. For instance, the Tn values may be inter-compared to determine the minimum Tn value. Also, it is noted that computing the AMI type indicator based on the minimum Tn value does not exclude that one or more of the other obtained Tn values, i.e., the Tn value(s) that are/is higher than the minimum value, is used for computing the AMI type indicator. In particular, the minimum Troponin value may be the minimum of a second subset of values of the plurality of Troponin values, wherein the second subset of values consists of values of the plurality of Troponin values that are obtained in a given time interval.

According to an embodiment, the method further comprises determining a rate of change of the Troponin concentration based on the first Troponin value and second Troponin value, and optionally one or more further Tn values, wherein the AMI type indicator is computed based on the determined rate of change of the Troponin concentration. The rate of change of Tn concentration is interchangeably used in the following with the rate of change of the Tn value and with Troponin rate.

For example, the rate of change of the Tn concentration may be provided by the difference of two Tn values or by the ratio between the difference of two Tn values and the difference between the time points at which said two Tn values are obtained. Similarly, a rate of change of the Tn concentration may be determined based on a plurality of more than two Tn values and used for computing the AMI type indicator. Also, it is noted that computing the AMI type indicator based on the rate of change of the Tn concentration does not exclude that the obtained Tn values and/or values computed based thereon, such as the maximum Tn value, the minimum Tn value and/or a mean Tn value, are used for computing the AMI type indicator.

According to an embodiment, the method further comprises determining a mean Troponin concentration or value based on the first Troponin value and second Troponin value, and optionally one or more further Tn values, wherein the AMI type indicator is computed based on the determined mean Troponin concentration. Likewise, a mean Tn concentration or value may be determined based on a plurality of more than two Tn values and used for computing the AMI type indicator. Also, it is noted that computing the AMI type indicator based on the mean Tn concentration or value does not exclude that the obtained Tn values and/or values computed based thereon, such as the maximum Tn value, the minimum Tn value and/or the rate of change of the Tn concentration, are used for computing the AMI type indicator. In particular, the mean Troponin concentration or value may be the mean of a third subset of values of the plurality of Troponin values, wherein the second subset of values consists of values of the plurality of Troponin values that are obtained in a given time interval.

According to an embodiment, the method further comprises determining a most recent Tn value based on the first Troponin value and second Troponin value, and optionally one or more further Tn values, wherein the AMI type indicator is computed based on the determined most recent Tn value. For instance, the Tn value with the most recent timestamp or having a timestamp closest to a time of computation of the AMI type indicator may be selected. Also, it is noted that computing the AMI type indicator based on the most recent Tn value does not exclude that the obtained Tn values and/or values computed based thereon, such as the maximum Tn value, the minimum Tn value, the mean value and/or the rate of change of the Tn concentration, are used for computing the AMI type indicator.

According to an embodiment, the subject data is indicative of a plurality of Troponin values determined for the subject at different times, for example including the first Tn value and second Tn value mentioned above. Therein, the method further comprises determining one or more of a maximum Troponin value of the plurality of obtained Troponin values, a minimum Troponin value of the plurality of obtained Troponin values, a most recent Tn value of the plurality of Tn values, a mean Tn value and a rate of change of the Troponin concentration. Therein, each of the plurality of Tn values may be determined for the subject at a respective time, wherein times for at least a subset of the Tn values may differ. For instance, a time series of two or more Tn values determined at different and/or consecutive times may be used to compute the AMI type indicator.

According to an embodiment, the subject data is indicative of a plurality of Troponin values determined for the subject at different times, such as for example the first Tn value and second Tn value mentioned above. Therein, the method further comprises determining one or both a maximum Troponin value of the plurality of obtained Troponin values, and a rate of change of the Tn concentration or value over time. In particular, the rate of change of the Tn concentration or value and the maximum Tn value may be used with the at least one Albumin value and the at least Eosinophil value to compute the at least one AMI type indicator. This parameter combination has been found to allow for an accurate differentiation between type 1 AMI and type 2 AMI.

According to an embodiment, the subject data is indicative of a plurality of Troponin values determined for the subject at different times, wherein the method further comprises determining a most recent Tn value among the plurality of Tn values, and optionally determining one or both a maximum Troponin value of the plurality of obtained Troponin values, and a rate of change of the Tn concentration or value over. In particular, the most recent Tn value may be used in combination with one or both the rate of change of the Tn concentration or value and the maximum Tn value to compute the at least one AMI type indicator.

According to an embodiment, computing the at least one AMI type indicator is based on one or more of the maximum Troponin value, the determined rate of change of the Troponin concentration, the at least one Albumin value, and the at least one Eosinophil value. In particular, using all of the maximum Tn value, the rate of change in Tn concentration, at least one Albumin value and at least one Eosinophil value were found to provide highly accurate results allowing to accurately determine the AMI type indicator that accurately reflects the actual type of AMI a subject is having. As noted above one or more of the Tn values, for example a most recent Tn value among the plurality of Tn values, may optionally be considered in addition or alternatively to one or both the maximum Tn value and the rate of change of the Tn value.

In an embodiment, the subject data is indicative of a plurality of Albumin values determined for the subject at different times, wherein the method further comprises determining one or more of a maximum Albumin value of the plurality of obtained Albumin values, a minimum Albumin value of the plurality of obtained Albumin values, a mean Albumin value, and a rate of change of the Albumin concentration. Further, the computation of the AMI indicator may optionally be based one or more of the maximum Albumin value, the minimum Albumin value, the mean Albumin value, and the rate of change of the Albumin concentration.

In an embodiment, the subject data is indicative of a plurality of Eosinophil values determined for the subject at different times, wherein the method further comprises determining one or more of a maximum Eosinophil value of the plurality of obtained Eosinophil values, a minimum Eosinophil value of the plurality of obtained Eosinophil values, a mean Eosinophil value and a rate of change of the Eosinophil concentration. Further, the computation of the AMI indicator may optionally be based one or more of the maximum Eosinophil value, the minimum Eosinophil value, the mean Eosinophil value, and the rate of change of the Eosinophil concentration.

According to an embodiment, the at least one Eosinophil value is indicative of an absolute concentration of Eosinophils in a blood sample of the subject. Alternatively or additionally, the at least one Eosinophil value is indicative of a fraction of Eosinophils in Leukocytes in a blood sample of the subject. For instance, one or both an absolute concentration or a relative count of Eosinophils with respect to Leukocytes may be determined in a measurement and used as Eosinophil value(s). The one or more Eosinophil values can be obtained by various hematology measurements, including measurements based on the Coulter principle, flowcytometry, fluorescence, light scattering measurements and others.

As mentioned above, one or more Tn values, one or more Albumin values, and one or more Eosinophil values, and optionally one or more other parameter values described herein, may be included in the subject data and used for the computation of one or more AMI type indicators. It is noted that all or a subset of a plurality of parameter values of a particular parameter can be used to compute the one or more AMI type indicators.

For example, the method can comprise determining the at least one AMI type indicator based on current subject data and past subject data, for example obtained within or over a time window of predetermined length. Alternatively or additionally, a predefined number of parameter values in the past may be considered, such as the last two, three, four or more parameter values of a particular parameter.

For instance, the method may comprise determining all parameter values falling within a predetermined period of time, for example preceding the time of computation of the AMI type indicator. This may include determining whether the timestamp of a parameter value is in the predetermined period of time. Optionally, one or more of the minimum value, the maximum value, the most recent value and/or a mean value of a plurality of parameter values of a particular parameter, including Troponin, and having timestamps within the predetermined period of time may be computed by the computing device and used to compute the at least one AMI type indicator.

In particular, at a given time point, the computing device and/or at least one ML model may use all subject data of at least one parameter, for instance including all Tn values, recorded in the immediate past preceding the time of computation of the at least one AMI type indicator within a predetermined period of time, such as 12 hours, 24 hours, 2 days, 3 days or more.

According to an embodiment, the method comprises obtaining and/or determining contextual information associated with the determined at least AMI type indicator. For example, the contextual information may include information or data indicative or reflective of a computational basis leading to the determined one or more AMI type indicators.

The contextual information may include data or information for interpreting the determined one or more AMI type indicators. Exemplary contextual information may include one or more notifications, messages or other informative means, which may optionally be provided at a user interface of the computing device. In an example, the contextual information may include an indication or information about the parameters and/or parameter values used for computing the one or more AMI type indicators, and optionally their contribution in the computation of the one or more AMI type indicators.

According to an embodiment, the subject data is indicative of at least one further hematology, vital and/or laboratory parameter selected from the group consisting of: MCHC (mean corpuscular hemoglobin concentration), age, gender, race, Lymphocyte fraction, Lymphocyte count, diastolic blood pressure, systolic blood pressure value, oxygen saturation, red blood count (RBC), white blood count (WBC), respiratory rate, minimum respiratory rate, maximum respiratory rate, hemoglobin, Blood Urea Nitrogen (BUN), C-Reactive Protein (CRP), body temperature, minimum body temperature, maximum body temperature, hematocrit, Red Cell Distribution Width (RDW), Monocyte Distribution Width (MDW).

As discussed above, the subject data may optionally include a plurality of Tn values and the method may comprise determining one or more of a rate of change of the Tn concentration or value over time, a maximum Tn value among the plurality of Tn values, and a most recent Tn value.

In the following, exemplary and non-limiting sets of parameter value combinations are provided, which have been found by the inventors to provide accurate AMI type indicators accurately reflecting the actual AMI type of the subject. For example, the at least one AMI type indicator may be based on the rate of change of the Tn concentration, the maximum Tn value, the at least one Albumin value, and the at least one Eosinophil value. Alternatively or additionally, the at least one AMI type indicator may be based on the rate of change of the Tn concentration, the maximum Tn value, the most recent Tn value, the at least one Albumin value, and the at least one Eosinophil value. Alternatively or additionally, the at least one AMI type indicator may be based on the rate of change of the Tn concentration, the maximum Tn value, the mean corpuscular hemoglobin concentration value (MCHC value), optionally the most recent Tn value, the at least one Albumin value, and the at least one Eosinophil value. Alternatively or additionally, the at least one AMI type indicator may be based on the rate of change of the Tn concentration, the maximum Tn value, the mean corpuscular hemoglobin concentration value (MCHC value), optionally one or both the most recent Tn value and a minimum Tn value, the at least one Albumin value, and the at least one Eosinophil value. Alternatively or additionally, the at least one AMI type indicator may be based on the rate of change of the Tn concentration, the maximum Tn value, the mean corpuscular hemoglobin concentration value (MCHC value), optionally one or both the most recent Tn value and a minimum Tn value, the at least one Albumin value, and the at least one Eosinophil value, and optionally an age value of the subject.

According to an embodiment, the computation of the at least one AMI type indicator is based on the rate of change of the Tn concentration, the maximum Tn value, the at least one Albumin value, the most recent Tn value, the at least one Eosinophil value, and the mean corpuscular hemoglobin concentration value (MCHC value).

According to an embodiment, the computation of the at least one AMI type indicator is based on the rate of change of the Tn concentration, the maximum Tn value, the at least one Albumin value, the most recent Tn value, the at least one Eosinophil value, the mean corpuscular hemoglobin concentration value (MCHC value), and an age value of the subject.

According to an embodiment, the computation of the at least one AMI type indicator is based on the rate of change of the Tn concentration, the maximum Tn value, the at least one Albumin value, the most recent Tn value, the at least one Eosinophil value, and the mean corpuscular hemoglobin concentration value (MCHC value), a minimum Tn value, and an age value of the subject.

According to an embodiment, the computation of the at least one AMI type indicator is based on the rate of change of the Tn concentration, the maximum Tn value, the at least one Albumin value, the most recent Tn value, the at least one Eosinophil value, and the mean corpuscular hemoglobin concentration value (MCHC value), a minimum Tn value, an age value of the subject, a Lymphocytes value, a minimum diastolic blood pressure value, and optionally a red blood count.

According to an embodiment, the computation of the at least one AMI type indicator is based on the rate of change of the Tn concentration, the maximum Tn value, the at least one Albumin value, the at least one Eosinophil value, and the mean corpuscular hemoglobin concentration value (MCHC value).

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), and an age value.

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), an age value, and a minimum diastolic blood pressure value.

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), an age value, a minimum diastolic blood pressure value, and a body temperature value.

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), an age value, a minimum diastolic blood pressure value, a body temperature value, and a minimum Tn value.

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), an age value, a minimum diastolic blood pressure value, a body temperature value, a minimum Tn value, and a red blood count.

According to an embodiment, the computation of the at least one AMI type indicator is based on the rate of change of the Tn concentration, the maximum Tn value, the at least one Albumin value, the at least one Eosinophil value, the mean corpuscular hemoglobin concentration value (MCHC value), an age value, a minimum diastolic blood pressure value, a body temperature value, a minimum Tn value, a red blood count, and a minimum respiratory rate value.

According to an embodiment, the method further comprises selecting at least one further hematology, vital and/or laboratory parameter, and determining the at least one AMI type indicator based on the at least one further hematology, vital and/or laboratory parameter.

The at least one further parameter may be selected among the group of parameters consisting of:
maximum Tn value;
rate of change of the Tn concentration or value;
MCHC value;
Age value;
Minimum diastolic blood pressure value;
Average diastolic blood pressure value;
Minimum body temperature value;
Average body temperature value;
Minimum Tn value;
Red blood count;
Minimum respiratory rate;
Most recent Tn value;
C-reactive protein;
Lymphocytes fraction;
BUN;
Lymphocyte count or value;
Body weight;
Maximum body weight;
D-dimer;
Hematocrit value;
Hemoglobin value; and
RDW.

One or more of the aforementioned parameter values may be used to compute the at least one AMI type indicator.

Optionally, one or more of the following further parameters may be selected among the group of parameters consisting of:
rate of change of the Tn concentration or value;
maximum Tn value;
Most recent Tn value;
MCHC value;
Age value;
Minimum Tn value;
Lymphocytes fraction;
Minimum diastolic blood pressure value;
Red blood count;
Minimum respiratory rate;
Hemoglobin value;
BUN;
C-reactive protein;
Minimum body temperature value;
Average body temperature;
Average diastolic blood pressure value;
Body weight;
Maximum body weight;
D-dimer;
Hematocrit value;
RDW; and
Lymphocyte count, value or fraction.

One or more of the aforementioned parameter values may be used to compute the at least one AMI type indicator.

According to a second aspect of the present disclosure, there is provided a computing device or system including one or more processors for data processing, wherein the computing device is configured to carry out steps of the method according to the first aspect of the present disclosure, as described hereinabove and hereinbelow.

The computing device may optionally comprise a data storage, for example storing at least a part of the subject data, data derived therefrom, the at least one AMI type indicator and/or information or data related thereto. Alternatively or additionally, software instructions or one or more computer programs may be stored at the data storage, which when executed by one or more processors of the computing device, may instruct the computing device to perform steps of the method according to the first aspect of the present disclosure, as described hereinabove and hereinbelow.

According to an embodiment, the computing device includes one or more machine learning models, for example implemented in a classifier circuitry or logic of the computing device, as described in more detail hereinabove and hereinbelow.

The computing device described herein may refer to any data processing device, including a standalone computing device or server and computing networks with a plurality of inter-operating computing devices, such as a cloud computing system or server system. Alternatively or additionally, the computing device may be embodied, at least in part, as mobile device, such as a smart phone a tablet computer, a notebook or the like.

The computing device may, in an example, comprise one or more communication interfaces configured to communicate with one or more remote devices, for example an external data storage or database, and/or one or more remote computing devices. For instance, at least a part of the subject data may be received via the communication interface from an external data storage. Alternatively or additionally, the computed at least one AMI type indicator or information related thereto may be transmitted to one or more remote devices or stored at the external data storage.

A third aspect of the present disclosure relates to a computer program, which, when executed by one or more processors of a computing device, instructs the computing device to perform steps of the method according to the first aspect of the present disclosure, as described hereinabove and hereinbelow.

A fourth aspect of the present disclosure relates to a computer-readable medium, for example a non-transitory computer-readable medium, storing a computer program, which, when executed by one or more processors of a computing device, instructs the computing device to perform steps of the method according to the first aspect of the present disclosure, as described hereinabove and hereinbelow. In particular, the computer-readable medium according to the fourth aspect of the present disclosure stores the computer program according to the third aspect of the present disclosure.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to exemplary embodiments which are illustrated in the attached drawings, wherein:

FIG. 1 shows a computing device or system for determining at least one AMI type indicator according to an exemplary embodiment;

FIG. 2 shows a flow chart illustrating a method of determining at least one AMI type indicator according to an exemplary embodiment; and

FIGS. 3a to 3e each show a receiver-operating-characteristic, ROC, curve illustrating methods of determining at least one AMI type indicator according to exemplary embodiments.

The figures are schematic only and not true to scale. In principle, identical or like parts are provided with identical or like reference symbols in the figures.

### DETAILED DESCRIPTION OF EXEMPLARY EMBODIMENTS

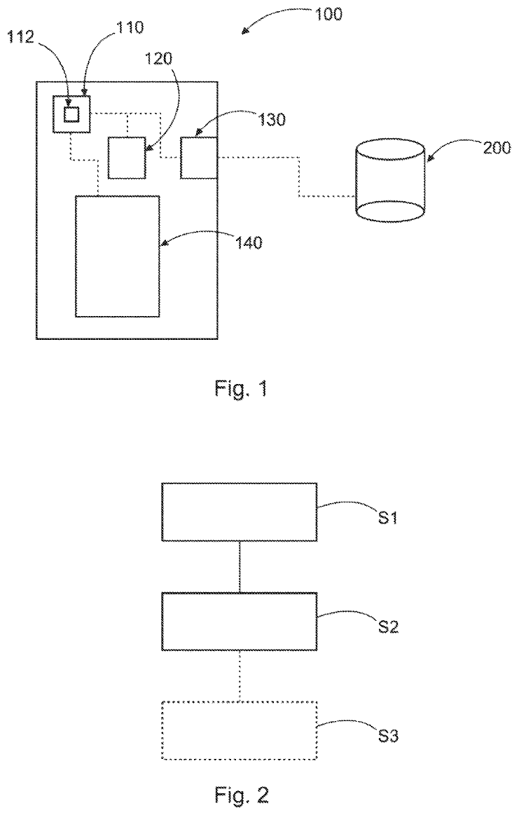

FIG. 1 shows a computing device or system 100, for example a clinical decision support system 100, configured to determine, for a patient, subject and/or individual, the at least one AMI type indicator indicative of a type of AMI the subject may have.

The computing device 100 comprises a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data and/or software instructions. For example, at least a subset of the subject data and/or the at least one AMI type indicator may be stored at the data storage 120.

The exemplary computing device 100 of FIG. 1 further comprises at least one communication circuitry or interface 130 for communicatively coupling the computing device 100 to one or more external data sources 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server or data processing device.

As discussed in detail hereinabove and hereinbelow, the computing device 100 may be configured to perform steps of the method of determining the at least one AMI type indicator based on subject data including at least Tn value, at least one Albumin value and at least Eosinophil value. Therein, the computing device 100 receives or obtains the subject data indicative of the at least one Tn value, the at least one Eosinophil value and the at least one Albumin value. Further, the computing device 100 is configured to determine, based on processing the received set of patient data, and/or compute the at least one AMI type indicator indicative of one or both a likelihood for the subject to have a type 1 AMI and a likelihood for the subject to have a type 2 AMI, as described in detail in the summary part.

The actual computation of the at least one AMI type indicator, the likelihood for the subject to have a type 1 AMI and/or the likelihood for the subject to have a type 2 AMI may be performed based on at least one Machine Learning, ML, model of the computing device 100. The ML model may comprise one or more of a trained logistic regression, a trained random forest, a trained gradient boosting algorithm, and a trained artificial neural network.

Exemplarily, the ML model comprises the XGBoost algorithm defined by Chen and Guestrin. A benefit of XGBoost algorithm is that it can deal with missing information without requiring data imputation.

Optionally, the subject data may comprise a plurality of Tn values determined at or associated with different times, and the computing device 100 may compute one or more of a maximum Tn value of the plurality of Tn values, a rate of change of the Tn value or concentration, a most recent Tn value and a minimum Tn value. Further optionally, a mean Tn value may be computed. One or more of the aforementioned calculated parameter values may be used to compute the at least one AMI type indicator. Further optionally, a plurality of Albumin values and/or Eosinophil values may be included in the subject data and corresponding parameter values may be computed and used to compute the AMI type indicator.

Optionally, the subject data is indicative of at least one further hematology, vital and/or laboratory parameter selected from the group consisting of: MCHC (mean corpuscular hemoglobin concentration), age, gender, race, Lymphocyte, minimum or maximum diastolic blood pressure, minimum or maximum systolic blood pressure value, (minimum or maximum) oxygen saturation, red blood count (RBC), white blood count (WBC), respiratory rate, minimum respiratory rate, maximum respiratory rate, hemoglobin, Blood Urea Nitrogen (BUN), C-Reactive Protein (CRP), body temperature, minimum body temperature, maximum body temperature, hematocrit, Red Cell Distribution Width (RDW), Monocyte Distribution Width (MDW). Any one or more of the aforementioned parameters may be considered in the computation of the at least one AMI type indicator.

Also, it is noted that a plurality of any one or more of the aforementioned parameters may be comprised in the subject data and further parameter values may be derived therefrom by the computing device. In particular, the computing device 100 may be configured to determine a minimum diastolic blood pressure value among a plurality of diastolic blood pressure values and compute the at least one AMI type indicator based thereon. Alternatively or additionally, the computing device 100 may be configured to determine a minimum respiratory rate value among a plurality of respiratory rate values and compute the at least one AMI type indicator based thereon. Alternatively or additionally, the computing device 100 may be configured to determine a minimum body temperature value among a plurality of body temperature values and compute the at least one AMI type indicator based thereon.

FIG. 2 shows a flow chart illustrating a computer-implemented method of determining at least one AMI type indicator according to an exemplary embodiment, for example using a computing device 100 as described with reference to FIG. 1.

Step S1 comprises obtaining, at a computing device 100, subject data associated with a subject suspected of having AMI. The subject data comprises a) at least one Troponin value indicative of a Troponin concentration determined for the subject, b) at least one Albumin value indicative of an Albumin concentration determined for the subject, and c) at least one Eosinophil value indicative of an Eosinophil concentration determined for the subject.

Step S2 comprises computing, based on the obtained subject data with the computing device 100, at least one AMI type indicator indicative of one or both a likelihood for the subject to have a type 1 AMI and a likelihood for the subject to have a type 2 AMI.

t is noted that one or more further optional steps, as described hereinabove in the summary part and with reference to FIG. 1, may be performed for determining the at least one AMI type indicator.

For example, in an optional step S3 the at least one AMI type indicator and/or information indicative of a likelihood for the subject having AMI type 1 and/or a likelihood for the subject having AMI type 2 may optionally be generated and potentially displayed at the user interface 140 of the computing device 100.

As mentioned above, the at least one AMI type indicator can be computed based on or using one or more ML models. The one or more machine learning models can be trained using a set of historical emergency department (ED) electronic health record (EHR) data from health care systems, e.g. across the USA, as training data (also referred to as training subject data). An inclusion criterion can be that the ED/EHR data include at least one Tn, value e.g. at least one hsTnI (high-sensitivity troponin I) value. The data can include de-identified demographic, clinical, and diagnostic information for the patient or subject in the encounter.

For example, the training data or training subject data may be obtained from the EHR data based on extracting data being associated with a particular subject, e.g. a subject diagnosed of AMI, and being indicative of one or more of the parameters considered as input for the one or more machine learning models, such as the at least one troponin value, the at least one Albumin value, the at least one Eosinophil and/or any other parameter described herein to be potentially usable for computing the at least one AMI type indicator. Each instance of patient or EHR data in the training data or training subject data may be associated with a respective known outcome in order to label the training data. For instance, the label can be a binary label wherein label 1 may equal or indicate AMI type 1 and label 0 may equal or indicate AMI type 2, or vice versa. In an example, the ML model can be trained to provide the likelihood of subject data provided as input data being associated with one of AMI type 1 and AMI type 2.

The EHR data may further include an indication about one or more diseases a patient suffered from in the past. For instance, diagnosis codes, such as according to the International Statistical Classification of Diseases and Related Health Problems, so-called ICD codes, can be stored in the EHR data. Based on the diagnosis codes, it can be determined whether an AMI occurred in a particular patient in the past, e.g. such that a corresponding label for AMI can be generated and included in the training subject data.

The training subject data obtained from the EHR data may optionally be cleaned for subsequent processing, for example based on removing erroneous data records. Further optionally, the training subject data may be split into a training dataset, which can be used for optimizing the one or more machine learning models, and a test dataset, which can be used for validation of the one or more machine learning models. For instance, model parameters of the one or more machine learning models can be tuned or adjusted using the training dataset to determine optimized model parameters, which may then be used to validate the one or more machine learning models using the test dataset. The process can be repeated iteratively, for example until the one or more machine learning models satisfy a target performance on the test dataset.

In the following, an exemplary implementation of a machine learning model and aspects of training such model are summarized. The machine learning model may include one or more of a trained logistic regression, a trained random forest, a trained gradient boosting algorithm, and a trained artificial neural network.

In particular, the method described herein may be implemented at least in part in a logistic regression ML model. Alternatively, a boosting technique may be utilized, such as XGBOOST, which however, is optional only.

When trained and/or during inference of the trained ML model, the at least one Tn value, the at least one Eosinophil value, and the at least one Albumin value of a subject can be provided as input. The at least one AMI type indicator may then be computed and a corresponding output may be generated, for example in the form of %-likelihood for AMI type 2. The AMI type indicator for AMI type 1 may also be computed as (100%-(%-likelihood for AMI type 2)).

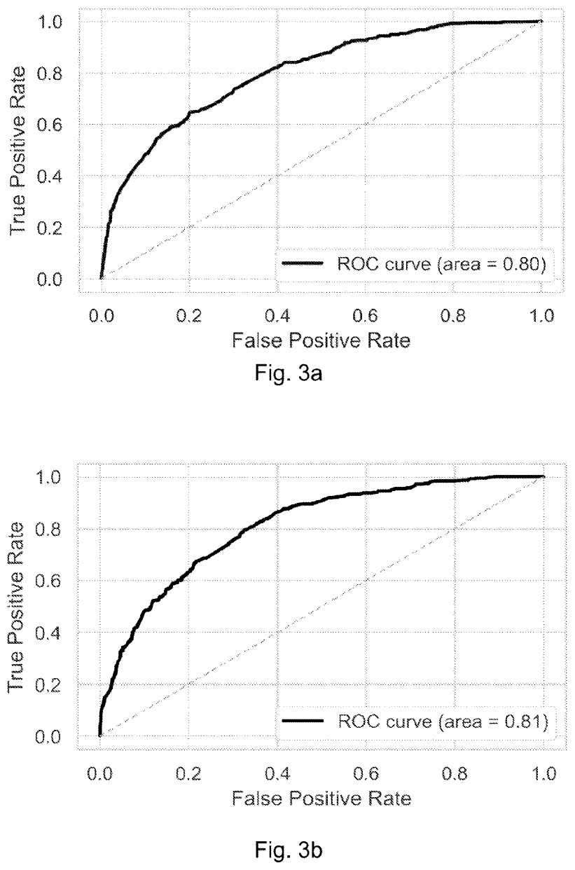

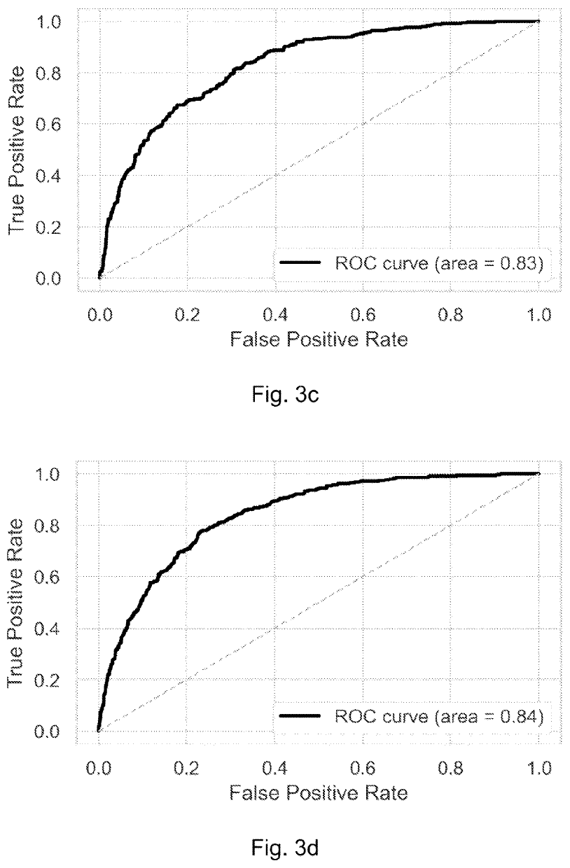

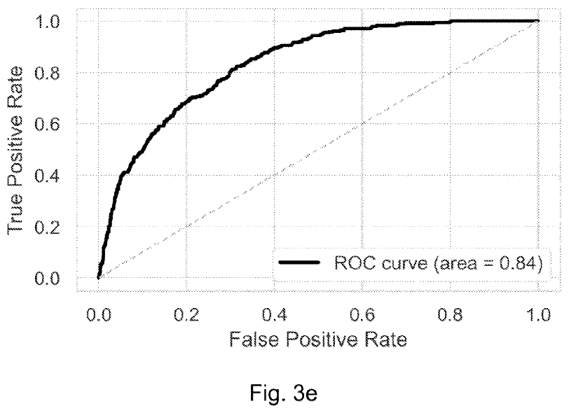

FIGS. 3a to 3e each show a receiver-operating-characteristic, ROC, curve illustrating methods of determining at least one AMI type indicator according to exemplary embodiments.

Generally, an ROC curve is a graph showing the performance of a classification model at all classification thresholds, wherein the True Positive Rate, TPR, is plotted versus the False Positive Rate, FPR. The TPR is defined as the number of True Positives (TP), i.e. an outcome where the ML model correctly predicts the positive class, divided by the sum of True Positives and False Negatives (FN), i.e. an outcome where the ML model incorrectly predicts the negative class: TPR=TP/(TP+FN). The FPR, on the other hand is defined as the number of False Positives (FP), i.e. an outcome where the ML model incorrectly predicts the positive class, divided by the sum of False Positives and True Negatives (TN), i.e. an outcome where the ML model correctly predicts the negative class: FPR=FP/(FP+TN).

FIGS. 3a to 3e show ROC curves for ML models considering a different number of input parameters or parameter values. Specifically, FIG. 3a shows the ROC curve for a ML model utilizing three parameters as input parameters, namely, the at least one Albumin value, the at least one Eosinophil value and the at least one Troponin value, which could be a first Tn value, a minimum, a maximum or average Tn value of a plurality of Tn values. FIG. 3b shows the ROC curve for a ML model utilizing in addition to the parameters considered in FIG. 3a, a second Troponin value, and a rate of change of the Tn concentration or Tn rate. FIG. 3c shows the ROC curve for a ML model utilizing in addition to the parameters considered in FIG. 3b, an average body temperature value, an MCHC value and a Hemoglobin value. FIG. 3d shows the ROC curve for a ML model utilizing in addition to the parameters considered in FIG. 3c, a value for C-reactive protein, a Hematocrit value, a minimum body temperature value, and an average diastolic blood pressure value. FIG. 3e shows the ROC curve for a ML model utilizing in addition to the parameters considered in FIG. 3d, a value for Eosinophil fraction, a minimum diastolic blood pressure, a minimum Tn value, a D-Dimer value, a Lymphocyte value, an age value, a maximum body weight value, a BUN value and an RDW value.

The two-dimensional area under the curve, AUC, under the ROC curve of FIGS. 3a to 3e provides an aggregate measure of the performance of the respective ML model. In the following Table, 1 the ROC-AUC values are provided for each of the ROC curves shown in FIGS. 3a to 3e.

TABLE 1

| Additive | | | | | | |
|---|---|---|---|---|---|---|
| Order | Parameter (value) | ROC-AUC | | | | |
| 1 | Albumin | 0.84 | 0.84 | 0.83 | 0.81 | 0.80 |
| 2 | Eosinophils | | | | | |
| 3 | Max. Troponin | | | | | |
| 4 | 2^{nd} Troponin | | | | | |
| 5 | Troponin rate | | | | | |
| 6 | Average body | | | | | |
| | temperature | | | | | |
| 7 | MCHC | | | | | |
| 8 | Hemoglobin | | | | | |
| 9 | C-reactive protein | | | | | |
| 10 | Hematocrit | | | | | |
| 11 | Minimum body | | | | | |
| | temperature | | | | | |
| 12 | Diastolic blood | | | | | |
| | pressure average | | | | | |
| 13 | Eosinophil fraction | | | | | |
| 14 | Diastolic blood | | | | | |
| | pressure minimum | | | | | |
| 15 | Minimum Troponin | | | | | |
| 16 | d-dimer | | | | | |
| 17 | Lymphocytes | | | | | |
| 18 | Age | | | | | |
| 19 | Maximum body | | | | | |
| | weight | | | | | |
| 20 | BUN | | | | | |
| 21 | RDW | | | | | |

As shown in above Table 1, the ROC-AUC value or integral of the ROC curve is 0.8 for the ROC curve shown in FIG. 3a for the ML model considering three parameters, 0.81 for the ROC curve shown in FIG. 3b for the ML model considering five parameters, 0.83 for the ROC curve shown in FIG. 3c for the ML model considering eight parameters, 0.84 for the ROC curve shown in FIG. 3d for the ML model considering twelve parameters, and 0.84 for the ROC curve shown in FIG. 3e for the ML model considering 21 parameters, as listed in the left column of Table 1. Hence, the performance of the ML model considering three parameters is already quite high with a value of 0.8, while the performance can be further increased or improved when considering one or more further vital, laboratory or hematology parameters, as listed in the left column of Table 1 or described herein as being usable to compute the at least one AMI type indicator.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A computer-implemented method of determining an acute myocardial infarction, AMI, type indicator, the method comprising:
obtaining, at a computing device, subject data associated with a subject suspected of having AMI, the subject data comprising:
a) at least one Troponin value indicative of a Troponin concentration determined for the subject,
b) at least one Albumin value indicative of an Albumin concentration determined for the subject,
c) at least one Eosinophil value indicative of an Eosinophil concentration determined for the subject; and
computing, based on the obtained subject data with the computing device, at least one AMI type indicator indicative of one or both a likelihood for the subject to have a type 1 AMI and a likelihood for the subject to have a type 2 AMI.

2. The method according to claim 1, wherein computing the at least one AMI type indicator further comprises:
classifying, by using the subject data, the subject into at least one AMI type class of a plurality of AMI type classes, the plurality of AMI classes, comprising a type 1 AMI class of
subjects suspected of having type 1 AMI and a type 2 AMI class of subjects suspected of having type 2 AMI.

3. The method according to claim 1, wherein the at least one AMI type indicator is indicative of a classification result of classifying the subject into at least one of a type 1 AMI class of subjects having type 1 AMI and a type 2 AMI class of subjects having type 2 AMI.

4. The method according to claim 1, wherein computing the at least one AMI type indicator includes:
computing the likelihood for the subject to have type 1 AMI and the likelihood for the subject to have type 2 AMI.

5. The method according to claim 1, wherein computing the at least one AMI type indicator is based on at least one machine learning model configured to receive and process input data associated with at least a subset of the subject data.

6. The method according to claim 5, wherein the at least one machine learning model comprises one or more of a trained logistic regression, a trained random forest, a trained gradient boosting algorithm, and a trained artificial neural network.

7. The method according to claim 5, wherein the at least one AMI type indicator is computed based on at least one machine learning model of the computing device configured to compute the at least one AMI type indicator based on a single or a plurality of Troponin value as input data.

8. The method according to claim 1, wherein obtaining the subject data includes:
obtaining a first Troponin value indicative of a first Troponin concentration determined for the subject at a first time; and
obtaining a second Troponin value for the subject indicative of a second Troponin concentration determined for the subject at a second time different than the first time; and
wherein the AMI type indicator is computed based on the first Troponin value and based on the second Troponin value.

9. The method according to claim 8, further comprising:
determining a maximum Troponin value among the obtained first Troponin value and second Troponin value;
wherein the AMI type indicator is computed based on the determined maximum Troponin value.

10. The method according to claim 8, further comprising:
determining a minimum Troponin value among the first Troponin value and second Troponin value; and
wherein the AMI type indicator is computed based on the determined minimum Troponin value.

11. The method according to claim 8, further comprising:
determining a rate of change of the Troponin concentration based on the first Troponin value and second Troponin value; and
wherein the AMI type indicator is computed based on the determined rate of change of the Troponin concentration.

12. The method according to claim 1, wherein the subject data is indicative of a plurality of Troponin values determined for the subject at different times;
wherein the method further comprises:
determining one or more of a maximum Troponin value of the plurality of obtained Troponin values, a minimum Troponin value of the plurality of obtained Troponin values, a most recent value of the plurality of Troponin values, and a rate of change of the Troponin concentration.

13. The method according to claim 12, wherein computing the at least one AMI type indicator is based on one or more of the maximum Troponin value, the determined rate of change of the Troponin concentration, the at least one Albumin value, and the at least one Eosinophil value.

14. The method according to claim 1, wherein the subject data is indicative of at least one further hematology, vital and/or laboratory parameter selected from the group consisting of: MCHC (mean corpuscular hemoglobin concentration), age, gender, race, Lymphocyte, diastolic blood pressure, systolic blood pressure value, oxygen saturation, red blood count (RBC), white blood count (WBC), respiratory rate, minimum respiratory rate, maximum respiratory rate, hemoglobin, Blood Urea Nitrogen (BUN), C-Reactive Protein (CRP), body temperature, minimum body temperature, maximum body temperature, hematocrit, Red Cell Distribution Width (RDW), Monocyte Distribution Width (MDW).

15. The method according to claim 1, further comprising:
selecting at least one further hematology, vital and/or laboratory parameter, and computing the at least one AMI type indicator based on the selected at least one further hematology, vital and/or laboratory parameter, selected among the group of parameters consisting of:
maximum Tn value;
rate of change of the Tn concentration or value;
MCHC value;
Age value;
Minimum diastolic blood pressure value;
Average diastolic blood pressure value;
Minimum body temperature value;
Average body temperature value;
Minimum Tn value;
Red blood count;
Minimum respiratory rate;
Most recent Tn value;
C-reactive protein;
Lymphocytes fraction;
BUN;
Lymphocyte count or value;
Hematocrit value;
Body weight;
Maximum body weight;
D-dimer;
Hemoglobin value; and
RDW.

16. A computing device including one or more processors for data processing, wherein the computing device is configured to carry out steps of the method according to claim 1.

17. (canceled)

18. A non-transitory computer-readable medium storing a computer program which, when executed by one or more processors of a computing device, instructs the computing device to perform steps of the method according to claim 1.

19. The method according to claim 4, wherein computing the at least one type one AMI type indicator includes comparing the likelihood for the subject to have type 1 AMI and the likelihood for the subject to have type 2 AMI with one another.

20. The method according to claim 4, wherein the computed at least one AMI type indicator is indicative of a difference between the computed likelihood for type 1 AMI and the computed likelihood for type 2 AMI.
